(19) United States
(12) Patent Application Publication
(10) Pub. No.: US 2024/0374747 A1
(43) Pub. Date: Nov. 14, 2024

(54) FUSION PROTEIN OF VHH ANTIBODY AND STREPTAVIDIN MUTANT

(71) Applicants: The University of Tokyo, Tokyo (JP); SAVID THERAPEUTICS INC., Tokyo (JP); TOHOKU UNIVERSITY, Miyagi (JP)

(72) Inventors: Toshiya TANAKA, Tokyo (JP); Tatsuhiko KODAMA, Tokyo (JP); Motomu KANAI, Tokyo (JP); Kenzo YAMATSUGU, Tokyo (JP); Toshifumi TATSUMI, Tokyo (JP); Akira SUGIYAMA, Tokyo (JP); Masanobu TSUKAGOSHI, Tokyo (JP); Masanori NAKAI, Tokyo (JP); Michael CHANSLER, Tokyo (JP); Hidetoshi TOKUYAMA, Miyagi (JP); Juri SAKATA, Miyagi (JP)

(73) Assignees: The University of Tokyo, Tokyo (JP); SAVID THERAPEUTICS INC., Tokyo (JP); TOHOKU UNIVERSITY, Miyagi (JP)

(21) Appl. No.: 18/619,761

(22) Filed: Mar. 28, 2024

(30) Foreign Application Priority Data
Mar. 29, 2023 (JP) 2023-053049

(51) Int. Cl. A61K 47/68; A61K 49/00; A61P 35/00; C07K 14/36; C07K 16/32

(52) U.S. Cl. CPC A61K 47/6855; A61K 47/6889; A61K 49/0032; A61P 35/00; C07K 14/36; C07K 16/32; C07K 2319/00

(57) ABSTRACT

A fusion protein of a molecule that recognizes cancer cells or the like and a mutant streptavidin, wherein the fusion protein is for use in the treatment or diagnosis of a cancer, is provided. In the fusion protein, a VHH antibody is bound, via a linker sequence, to the N-terminal side and/or C-terminal side of the amino acid sequence as set forth in SEQ ID NO: 1, provided that the C-terminal amino acid sequence consisting of Pro-Ser-Ala-Ala-Ser-His-His-His-His-His-His may be partially or entirely deleted.

### TECHNICAL FIELD

The present invention relates to a fusion protein of a VHH antibody and a mutant streptavidin and the use thereof.

### BACKGROUND ART

Avidin and biotin, or streptavidin and biotin have an extremely high affinity between them (Kd=10⁻¹⁵ to 10⁻¹⁴ M). This is one of the extremely strong interactions between two biomolecules. At present, the interaction between avidin/streptavidin and biotin has been widely applied in the field of biochemistry, molecular biology, or medicine. A drug delivery method and a pretargeting method have been devised, in which high binding ability between avidin/streptavidin and biotin is combined with an antibody molecule. In connection with these studies, a mutant streptavidin with a reduced affinity for natural biotin and a biotin modified dimer having a high affinity for the mutant streptavidin with a low affinity for natural biotin are reported in Patent Document 1.

In addition, Patent Document 2 describes a fusion protein, in which an antigen-binding molecule having a molecular weight of 20,000 or less is bound, via a linker sequence, to the N-terminal side and/or C-terminal side of a mutant streptavidin.

### PRIOR ART DOCUMENTS

### Patent Documents

[Patent Document 1] International Publication WO2015/125820
[Patent Document 2] International Publication WO2022/203000

### SUMMARY OF INVENTION

### Object to be Solved by the Invention

It is an object of the present invention to provide a fusion protein of a molecule that recognizes cancer cells or the like and a mutant streptavidin, wherein the fusion protein is used to treat or diagnose cancer. It is another object of the present invention to provide a means for treating cancer or a means for diagnosing cancer, in which the above-described fusion protein is used.

### Means for Solving the Object

As a result of intensive studies directed towards achieving the above-described objects, the present inventor has selected a VHH antibody, as a molecule that recognizes cancer cells, and then, the present inventor has prepared a fusion protein of the aforementioned VHH antibody and a mutant streptavidin. The present inventor has found that the proliferation of cells can be suppressed by photoimmunotherapy using the above-described fusion protein and a conjugate of a biotin modified dimer and a photosensitizer. In addition, the present inventor has found that the proliferation of cells can be suppressed and also the tumor proliferation can be suppressed in vivo, by administering the above-described fusion protein and a conjugate of a biotin modified dimer and an anti-cancer drug. The present invention has been completed based on these findings.

Specifically, according to the present invention, the following inventions are provided.
<1> A fusion protein, wherein a VHH antibody is bound, via a linker sequence, to the N-terminal side and/or C-terminal side of the amino acid sequence as set forth in SEQ ID NO: 1, provided that the C-terminal amino acid sequence consisting of Pro-Ser-Ala-Ala-Ser-His-His-His-His-His-His may be partially or entirely deleted.
<2> The fusion protein according to <1>, which has the VHH antibody, the linker sequence, and the amino acid sequence as set forth in SEQ ID NO: 1, provided that the C-terminal amino acid sequence consisting of Pro-Ser-Ala-Ala-Ser-His-His-His-His-His-His may be partially or entirely deleted, in this order from the N-terminal side to the C-terminal side.
<3> The fusion protein according to <1> or <2>, wherein the VHH antibody is an antibody that binds to an antigen expressing in a cancer cell.
<4> The fusion protein according to any one of <1> to <3>, wherein the VHH antibody is an antibody that binds to Her2.
<5> The fusion protein according to any one of <1> to <4>, wherein the VHH antibody has the amino acid sequence as set forth in SEQ ID NO: 2.
<6> The fusion protein according to any one of <1> to <5>, wherein the linker sequence consists of a glycine residue(s) and a serine residue(s) and the number of the amino acid residues is 5 to 25.
<7> The fusion protein according to any one of <1> to <6>, wherein the linker sequence is an amino acid sequence represented by [(Gly)ₘ-Ser]ₙ wherein m represents an integer of 1 to 10, and n represents an integer of 1 to 5.
<8> The fusion protein according to any one of <1> to <7>, which has the amino acid sequence as set forth in SEQ ID NO: 3, provided that the C-terminal amino acid sequence consisting of Pro-Ser-Ala-Ala-Ser-His-His-His-His-His-His may be partially or entirely deleted.
<9> A nucleic acid encoding the fusion protein having the amino acid sequence as set forth in SEQ ID NO: 3, provided that the C-terminal amino acid sequence consisting of Pro-Ser-Ala-Ala-Ser-His-His-His-His-His-His may be partially or entirely deleted.
<10> A cancer therapeutic agent or a cancer diagnostic agent, comprising the fusion protein according to any one of <1> to <8>.
<11> A kit for treating or diagnosing cancer, comprising (1) the fusion protein according to any one of <1> to <8>, and (2) a conjugate of a compound represented by the following formula (1) or a salt thereof, and a diagnostic substance or a therapeutic substance:

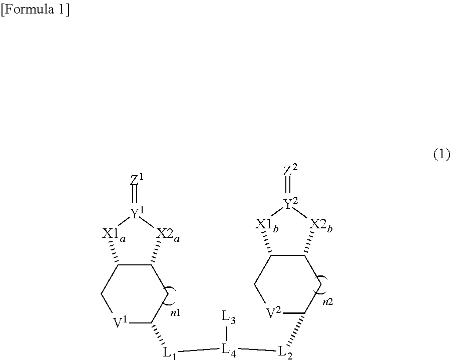

wherein
X1a, X1b, X2a and X2b each independently represent O or NH,
Y¹ and Y² each independently represent C or S,
Z¹ and Z² each independently represent O, S or NH,
V¹ and V² each independently represent S or S⁺—O⁻, n1 and n2 each independently represent an integer of 0 or 1,
L₁ and L₂ each independently represent a divalent linking group,
L₃ represents a group comprising a functional group capable of binding to the diagnostic substance or the therapeutic substance at the terminus thereof, and
L₄ represents a trivalent linking group.
<12> The kit according to <11>, wherein the diagnostic substance or the therapeutic substance is a phthalocyanine dye or an anti-cancer drug.
<13> A method for producing the fusion protein according to any one of <1> to <8>, comprising a step of allowing a nucleic acid encoding the fusion protein according to any one of <1> to <8> to express in a host.
<14> The method according to <13>, wherein the fusion protein is allowed to express in a bacterial inclusion body and is then recovered.

### Advantageous Effects of Invention

By using the fusion protein of the present invention consisting of a VHH antibody and a mutant streptavidin, for example, the proliferation of cancer cells can be suppressed.

### BRIEF DESCRIPTION OF DRAWINGS

FIGS. 1A and 1B show the map of the expression vector used to produce Anti-HER2 VHH-Cupid (FIG. 1A) and the results of the confirmation of the expression of Anti-HER2 VHH-Cupid (FIG. 1B)

FIG. 2 shows the results of confirmation of cytotoxic activity by using Anti-HER2 VHH-Cupid and the photosensitizer Psyche.

FIG. 3 shows the results of confirmation of cytotoxic activity by using Anti-HER2 VHH-Cupid and the anti-cancer drug Psyche (Psyche-Duocarmycin).

FIG. 4A shows the results of an in vivo experiment in xenograft model mouse using Anti-HER2 VHH-Cupid and Psyche-Duocarmycin and FIG. 4B shows the results per individual of an in vivo experiment in a xenograft mouse model using Anti-HER2 VHH-Cupid and Psyche-Duocarmycin.

FIG. 5 shows the results of an in vivo experiment in xenograft model mouse using Anti-HER2 VHH-Cupid and Psyche-Duocarmycin.

FIG. 6 shows the results of confirmation of the expression of Anti-HER2 VHH-Cupid.

FIG. 7 shows the results of study for pH conditions of dialysis buffer optimal for tetramer formation of Anti-HER2 VHH-Cupid.

FIGS. 8A-8C show the chromatogram as the result of the size exclusion chromatography for Anti-HER2 VHH-Cupid (FIG. 8A), and the electrophoretic diagram of sample fractions from each retention time (FIG. 8B), and the results of confirmation of purified tetramer (FIG. 8C).

FIG. 9 shows the results of confirmation of interaction between Anti-HER2 VHH-Cupid and Psyche-Duocarmycin.

FIG. 10 shows the results of confirmation of interaction between Anti-HER2 VHH-Cupid and extra-cellular domain of HER2 (HER2 ECD).

FIG. 11 shows t the results of confirmation of cellular internalization activity of Anti-HER2 VHH-Cupid and the fluorescent dye Psyche (Psyche-FITC).

FIG. 12 shows the results of confirmation of cytotoxic activity by using Anti-HER2 VHH-Cupid and Psyche-Duocarmycin.

FIGS. 13A-13C show the results of an in vivo experiment for tumor proliferation suppressing effect by the interval between repeated administrations in xenograft model mouse using Anti-HER2 VHH-Cupid and Psyche-Duocarmycin. FIG. 13A shows the results for the long interval group, and FIG. 13B shows the results for the short interval group, and FIG. 13C shows the results of control group.

FIG. 14 shows the results of pathological analysis after the in vivo experiment for tumor proliferation suppressing effect by the interval between repeated administrations in xenograft model mouse using Anti-HER2 VHH-Cupid and Psyche-Duocarmycin.

### EMBODIMENT OF CARRYING OUT THE INVENTION

Hereinafter, the present invention will be described in more detail.

### <Fusion Protein of VHH Antibody and Mutant Streptavidin>

The fusion protein of the present invention is a fusion protein, in which a VHH antibody is bound, via a linker sequence, to the N-terminal side and/or C-terminal side of the amino acid sequence as set forth in SEQ ID NO: 1 (provided that the C-terminal amino acid sequence consisting of Pro-Ser-Ala-Ala-Ser-His-His-His-His-His-His may be partially or entirely deleted). When the VHH antibodies are bound, via linker sequences, to both of the N-terminal side and/or C-terminal side of the amino acid sequence as set forth in SEQ ID NO: 1 (provided that the C-terminal amino acid sequence consisting of Pro-Ser-Ala-Ala-Ser-His-His-His-His-His-His may be partially or entirely deleted), the VHH antibodies may be identical to or different from each other.

Preferably, the fusion protein of the present invention is a fusion protein having the VHH antibody, the linker sequence, and the amino acid sequence as set forth in SEQ ID NO: 1 (provided that the C-terminal amino acid sequence consisting of Pro-Ser-Ala-Ala-Ser-His-His-His-His-His-His may be partially or entirely deleted) in this order from the N-terminal side to the C-terminal side.

The amino acid sequence as set forth in SEQ ID NO: 1 is the amino acid sequence of a mutant streptavidin, and specifically, this mutant streptavidin is the mutant streptavidin LISA314-V2122 described in Example 3 of International Publication WO2015/125820 (SEQ ID NO: 4 of International Publication WO2015/125820) (SEQ ID NO: 1 in the description of the present application).

As described above, the fusion protein of the present invention is a fusion protein of a VHH antibody and a mutant streptavidin. The fusion protein of the present invention forms a tetramer as a result of the affinity between the two amino acid sequences as set forth in SEQ ID NO: 1. When the protein having the amino acid sequence described in Example 2 is, for example, used as a VHH antibody, the molecular weight of the tetramer formed by the fusion protein of the present invention is approximately 110 kDa. When the fusion protein as used in the present invention is administered to a living body to treat cancer, it is important to simultaneously achieve the following points: favorable tumor uptake; good clearance rate; and favorable tumor penetration. It is conceived that the above-described molecular weight (approximately 110 kDa) of the present invention is a molecular weight, with which the above-described three parameters can be simultaneously achieved.

VHH antibody (Variable domain of Heavy chain of Heavy chain antibody) is a natural single-domain antibody with low-molecular weight derived from a variable region of a specific antibody (heavy chain antibody) found in the serum of camelids such as alpacas. The VHH antibodies have high stability against temperature and pH and can be produced at low cost using microorganism. The molecular weight of VHH antibodies is approximately 15,000 (15 kDa). The VHH antibodies are single-stranded proteins, and so it is easy to modify their functions by protein engineering or chemical modification. There are three complementarity-determining regions (CDRs) in VHH antibodies, CDR1, CDR2, and CDR3. Among these, CDR3 in the VHH antibodies is characterized by a length of 24 amino acid residues, compared to IgG antibodies, which have a length of 7 amino acid residues.

The antigen recognized by a VHH antibody is not particularly limited. The antigen is preferably an antigen that is expressed in cancer cells. Examples of the antigen that is specifically expressed in cancer may include the following antigens:

Epiregulin (EREG), ROBO 1, 2, 3 and 4, 1-40-β-amyloid, 4-1BB, 5AC, 5T4, ACVR2B, adenocarcinoma antigen, α-fetoprotein, angiopoetin 2, anthrax toxin, AOC3 (VAP-1), B-lymphoma cells, B7-H3, BAFF, β amyloid, C242 antigen, C5, CA-125, carbonic anhydrase 9 (CA-IX), cardiac myosin, CCL11 (eotaxin-1), CCR4, CCR5, CD11, CD18, CD125, CD140a, CD147 (basigin), CD147 (basigin), CD15, CD152, CD154 (CD40L), CD154, CD19, CD2, CD20, CD200, CD22, CD221, CD23 (IgE receptor), CD25 (IL-2 receptor a chain), CD28, CD3, CD30 (TNFRSF8), CD33, CD37, CD38 (cyclic ADP ribose hydrolase), CD4, CD40, CD41 (integrin α-IIb), CD44 v6, CD5, CD51, CD52, CD56, CD6, CD70, CD74, CD79B, CD80, CEA, CFD, ch4D5, CLDN18.2, Clostridium difficile, clumping factor A, CSF2, CTLA-4, cytomegalovirus, cytomegalovirus glycoprotein B, DLL4, DR5, E. coli Shiga toxin type 1, E. coli Shiga toxin type 2, EGFL7, EGFR, endotoxin, EpCAM, episialin, ERBB3, Escherichia coli, F protein of respiratory syncytial virus, FAP, fibrin II β chain, fibronectin extra domain-B, folate receptor 1, Frizzled receptor, GD2, GD3 ganglioside, GMCSF receptor a chain, GPNMB, hepatitis B surface antigen, hepatitis B virus, HER1, HER2/neu, HER3, HGF, HIV-1, HLA-DRβ, HNGF, Hsp90, human β amyloid, human scatter factor receptor kinase, human TNF, ICAM-1 (CD54), IFN-α, IFN-γ, IgE, IgE Fc region, IGF-1 receptor, IGF-I, IgG4, IGHE, IL-1β, IL-12, IL-13, IL-17, IL-17A, IL-22, IL-23, IL-4, IL-5, IL-6, IL-6 receptor, IL-9, ILGF2, influenza A hemagglutinin, insulin-like growth factor I receptor, integrin α4, integrin α4β7, integrin α5β1, integrin α7β7, integrin αIIbβ3, integrin αvβ3, integrin γ induced protein, interferon receptor, interferon α/β receptor, ITGA2, ITGB2 (CD18), KIR2D, L-selectin (CD62L), Lewis-Y antigen, LFA-1 (CD11a), lipoteichoic acid, LOXL2, LTA, MCP-1, mesothelin, MS4A1, MUC1, mucin CanAg, myostatin, N-glycolylneuraminic acid, NARP-1, NCA-90 (granulocyte antigen), NGF, NOGO-A, NRP1, Oryctolagus cuniculus, OX-40, oxLDL, PCSK9, PD-1, PDCD1, PDGF-R α, phosphatidylserine, prostate cancer cells, Pseudomonas aeruginosa, Rabies virus glycoprotein, RANKL, respiratory syncytial virus, RHD, Rh (Rhesus) factor, RON, RTN4, sclerostin, SDC1, selectin P, SLAMF7, SOST, sphingosine-1-phosphate, TAG-72, TEM1, tenascin C, TFPI, TGFβ1, TGFβ2, TGF-β, TNF-α, TRAIL-R1, TRAIL-R2, tumor antigen CTAA16.88, MUC1 tumor-specific glycosylation, TWEAK receptor, TYRP1 (glycoprotein 75), VEGF-A, VEGFR-1, VEGFR2, vimentin, and VWF.

Among the above-described antigens, HER2 is particularly preferable.

An example of the VHH antibody may be a VHH antibody, which binds to HER2. An example of a such VHH antibody may be a protein having the amino acid sequence as set forth in SEQ ID NO: 2.

The linker sequence is not particularly limited, as long as the effects of the present invention can be achieved. The number of amino acids in the linker sequence is preferably 5 to 25 amino acids, more preferably 10 to 25 amino acids, and further preferably 15 to 20 amino acids.

A specific example of the linker sequence may be a sequence consisting of glycine residues and serine residues. As such a linker sequence, for example, an amino acid sequence represented by [(Gly)ₘ-Ser]ₙ (wherein m represents an integer of 1 to 10, and n represents an integer of 1 to 5) can be used. A specific example of the linker sequence may be Gly-Gly-Gly-Gly-Gly-Ser-Gly-Gly-Gly-Gly-Gly-Ser-Gly-Gly-Gly-Gly-Gly-Ser, but is not particularly limited thereto.

A specific example of the fusion protein of the present invention may be a fusion protein having the amino acid sequence as set forth in SEQ ID NO: 3 (provided that the C-terminal amino acid sequence consisting of Pro-Ser-Ala-Ala-Ser-His-His-His-His-His-His may be partially or entirely deleted).

According to the present invention, a nucleic acid (for example, DNA) that encodes the above-described fusion protein of the present invention is further provided. A specific example of the nucleic acid of the present invention may be a nucleic acid that encodes the fusion protein having the amino acid sequence as set forth in SEQ ID NO: 3 (provided that the C-terminal amino acid sequence consisting of Pro-Ser-Ala-Ala-Ser-His-His-His-His-His-His may be partially or entirely deleted). One example of the nucleic acid of the present invention may be a nucleic acid having the nucleotide sequence as set forth in SEQ ID NO: 4.

A nucleic acid (for example, DNA) encoding the fusion protein of the present invention can be used by being incorporated into a vector. In order to produce the fusion protein of the present invention, a nucleic acid encoding the fusion protein of the present invention is incorporated into an expression vector, and a host is then transformed with this expression vector, so that the fusion protein of the present invention can be expressed in the host. According to the present invention, there is a method for producing the fusion protein of the present invention, comprising a step of allowing a nucleic acid encoding the fusion protein of the present invention to express in a host. Preferably, the fusion protein can be expressed in a bacterial inclusion body and recovered therefrom.

When Escherichia coli is used as a host, the vector preferably has a replication origin (ori) and also has a gene for selecting the transformed host (e.g. a drug-resistance gene that is resistant to drugs, such as ampicillin, tetracycline, kanamycin or chloramphenicol, etc.). Moreover, an expression vector preferably has a promoter capable of efficiently expressing the mutant streptavidin of the present invention in a host, such as a lacZ promoter or a T7 promoter. Examples of such a vector include an M13 vector, a pUC vector, pBR322, pBluescript, pCR-Script, pGEX-5X-1 (Pharmacia), “QIAexpress system” (QIAGEN), pEGFP, and pET (in this case, BL21 that expresses T7 RNA polymerase is preferably used as a host).

A vector can be introduced into a host cell by applying a calcium chloride method or an electroporation method, for example. Further, a sequence that encodes a tag for improving solubility, such as glutathione S-transferase, thioredoxin or a maltose-binding protein, may be added. Still further, a sequence that encodes a tag designed for facilitating purification, such as a polyhistidine tag, a Myc epitope, a hemagglutinin (HA) epitope, a T7 epitope, an Xpress tag, a FLAG tag or other known tag sequences, may also be added.

Other than Escherichia coli, examples of the expression vector include: mammal-derived expression vectors (for example, pcDNA3 (manufactured by Invitrogen), pEGF-BOS (Nucleic Acids. Res. 1990, 18(17), p. 5322), pEF and pCDM8); insect cell-derived expression vectors (for example, “Bac-to-BAC baculovirus expression system” (manufactured by Gibco-BRL) and pBacPAK8); plant-derived expression vectors (for example, pMH1 and pMH2); animal virus-derived expression vectors (for example, pHSV, pMV and pAdexLcw); retrovirus-derived expression vectors (for example, pZIPneo); yeast-derived expression vectors (for example, “Pichia Expression Kit” (manufactured by Invitrogen), pNV11 and SP-Q01); and Bacillus subtilis-derived expression vectors (for example, pPL608 and pKTH50).

When the expression of the present fusion protein in an animal cell such as a CHO cell, a COS cell or an NIH3T3 cell is intended, it is essential for the expression vector to have a promoter necessary for the expression of the fusion protein in such an animal cell, such as an SV40 promoter (Mulligan et al., Nature (1979) 277, 108), an MMLV-LTR promoter, an EF1α promoter (Mizushima et al., Nucleic Acids Res. (1990) 18, 5322) or a CMV promoter. It is more preferable if the expression vector has a gene for selecting the transformation of a cell (for example, a drug-resistance gene capable of determining transformation with the use of drugs (neomycin, G418, etc.)). Examples of a vector having such properties include pMAM, pDR2, pBK-RSV, pBK-CMV, pOPRSV and pOP13.

The type of a host cell, into which the vector is introduced, is not particularly limited. Either prokaryotes or eukaryotes may be used. It is possible to use Escherichia coli or various types of animal cells, for example.

In the case of using a eukaryotic cell, for example, an animal cell, a plant cell or a fungal cell can be used as a host. Examples of an animal cell that can be used herein include: mammalian cells such as a CHO cell, a COS cell, a 3T3 cell, a HeLa cell or a Vero cell; and insect cells such as Sf9, Sf21 or Tn5. When the expression of a large amount of the fusion protein in an animal cell is intended, a CHO cell is particularly preferable. A vector can be introduced into a host cell by a calcium phosphate method, a DEAE-dextran method, a method using cationic ribosome DOTAP (manufactured by Boehringer Mannheim), an electroporation method, a lipofection method or the like.

As a plant cell, a cell from Nicotiana tabacum is known as a protein-producing system, for example. These cells may be subjected to callus culture. Examples of a known fungal cell include: yeast cells including genus Saccharomyces such as Saccharomyces cerevisiae; and filamentous fungi including genus Aspergillus such as Aspergillus niger.

Examples of a prokaryotic cell that can be used herein include Escherichia coli (E. coli), such as JM109, DH5α or HB101. Moreover, Bacillus subtilis has been known.

These cells are transformed with the nucleic acid of the present invention, and the transformed cells are then cultured in vitro, so as to obtain the fusion protein of the present invention. The culture can be carried out in accordance with a known culture method. Examples of a culture solution of animal cells that can be used herein include DMEM, MEM, RPMI1640, and IMDM. During the culture, a serum infusion such as fetal calf serum (FCS) may be used in combination, or serum free culture may also be carried out. The pH applied during the culture is preferably approximately pH 6 to 8. The culture is generally carried out at a temperature of approximately 30° C. to 40° C. for approximately 15 to 200 hours. As necessary, medium replacement, ventilation and stirring are carried out. Furthermore, growth factors may also be added to promote the growth of cells.

### <Cancer Therapeutic Agent or Cancer Diagnostic Agent>

The fusion protein of the present invention is useful as a cancer therapeutic agent or a cancer diagnostic agent.

According to the present invention, provided is a kit for treating or diagnosing cancer, comprising (1) the fusion protein of the present invention, and (2) a conjugate of a compound represented by a formula (1) as shown below or a salt thereof, and a diagnostic substance or a therapeutic substance.

When a VHH antibody that is bound to an antigen existing in a cancer cell is used as such an VHH antibody, the fusion protein of the present invention is administered to a patient, so that a mutant streptavidin can be accumulated specifically into cancer cells. Subsequently, by administering a conjugate of a biotin modified dimer having an affinity for the mutant streptavidin and a diagnostic substance or a therapeutic substance to the patient, it becomes possible to accumulate the diagnostic substance or the therapeutic substance precisely into the cancer cells.

Otherwise, a complex is prepared by binding the “fusion protein of the present invention” with the “conjugate of a biotin modified dimer having an affinity for a mutant streptavidin and a diagnostic substance or a therapeutic substance,” and the thus prepared complex can be administered to the patient.

### <Biotin Modified Dimer>

The biotin modified dimer is a compound represented by the following formula (1) or a salt thereof, and is preferably a compound represented by the following formula (2) or a salt thereof. As such a biotin modified dimer, the compound described in International Publication WO2015/125820 can be used.

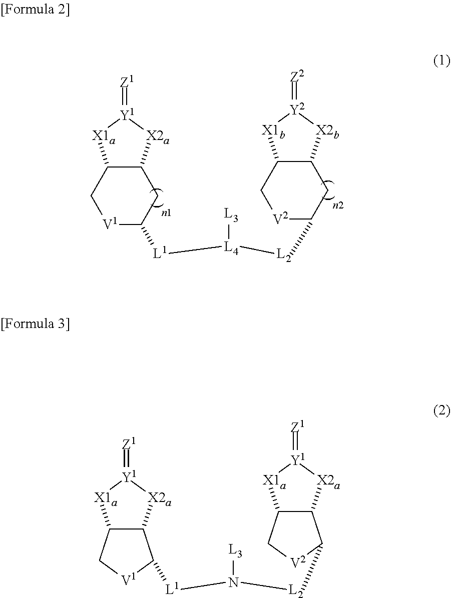

wherein
X1a, X1b, X2a and X2b each independently represent O or NH,
Y¹ and Y² each independently represent C or S,
Z¹ and Z² each independently represent O, S or NH,
V¹ and V² each independently represent S or S⁺—O⁻, n1 and n2 each independently represent an integer of 0 or 1,
L₁ and L₂ each independently represent a divalent linking group,
L₃ represents a group comprising a functional group capable of binding to the diagnostic substance or the therapeutic substance (for example, a phthalocyanine dye) at the terminus thereof, and
L₄ represents a trivalent linking group.

In the formula (1) and the formula (2), the portions represented by the following structures:

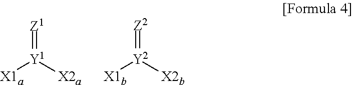

are preferably any one of the following portions, but are not limited thereto:

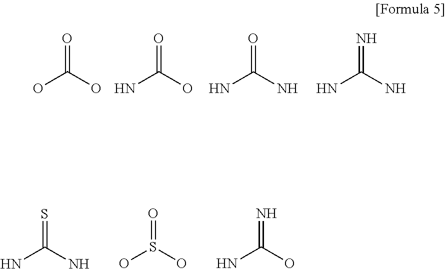

X1a, X1b, X2a and X2b preferably represent NH; Y¹ and Y² preferably represent C; Z¹ and Z² preferably represent NH; and V¹ and V² preferably represent S.

L₁ and L₂ each independently represent a divalent linking group consisting of a combination of groups selected from —CONH—, —NHCO—, —COO—, —OCO—, —CO—, —O—, and an alkylene group containing 1 to 10 carbon atoms.

Preferably, L₁ and L₂ each independently represent a divalent linking group consisting of a combination of groups selected from —CONH—, —NHCO—, —O—, and an alkylene group containing 1 to 10 carbon atoms.

Preferably, L₁ and L₂ each independently represent a divalent linking group consisting of a combination of groups selected from —CONH—, —NHCO—, and an alkylene group containing 1 to 10 carbon atoms.

L₄ represents a trivalent linking group, and is preferably the following:

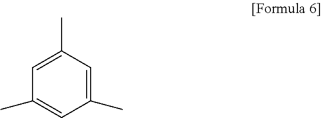

or,

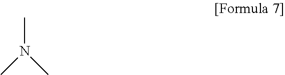

(which is a benzene-derived trivalent linking group or a nitrogen atom).

L₃ is preferably a group consisting of a combination of groups selected from —CONH—, —NHCO—, —COO—, —OCO—, —CO—, —O—, and an alkylene group containing 1 to 10 carbon atoms, and further comprising an amino group at the terminus thereof.

### <Conjugate of Biotin Modified Dimer and Diagnostic Substance or Therapeutic Substance>

By binding a diagnostic substance or a therapeutic substance to a biotin modified dimer, a conjugate of the biotin modified dimer and the diagnostic substance or the therapeutic substance can be prepared. Examples of the diagnostic substance or the therapeutic substance may include a fluorochrome, a chemiluminescent agent, a radioisotope, a sensitizer consisting of a metal compound or the like, a neutron-capturing agent consisting of a metal compound or the like, a phthalocyanine dye, a low-molecular-weight compound (e.g. anti-cancer drug), micro- or nano-bubbles, and a protein. Preferably, a phthalocyanine dye or a low-molecular-weight compound (e.g. anti-cancer drug) can be used.

As an example of anti-cancer drug, alkylating agents, antimetabolites, microtubule inhibitors, antibiotic anti-cancer agents, topoisomerase inhibitors, platinum drugs, molecularly targeting drugs, hormonal agents or biologics may be used. As an example of alkylating agents, nitrogen mustard anti-cancer drugs such as cyclophosphamide, nitrosourea anti-cancer drugs such as ranimustine, dacarbazine may be used. As an example of antimetabolites, 5-FU, UFT, carmofur, capecitabine, tegafur, TS-1, gemcitabine and cytarabine may be used. As an example of microtubule inhibitors, alkaloid anti-cancer drugs such as vincristine, taxane anti-cancer drugs such as docetaxel and paclitaxel may be used. As an example of antibiotic anti-cancer agents, duocarmycins, mitomycin C, doxorubicin, epirubicin, daunorubicin, and bleomycin may be used. Duocarmycins can be duocarmycin derivative described in International Publication WO2021/215534. As an example of topoisomerase inhibitors, irinotecan and nogitecan having topoisomerase I inhibitory activity, etoposide having topoisomerase II inhibitory activity may be used. As an example of platinum drugs, cisplatin, paraplatin, nedaplatin and oxaliplatin may be used. As an example of molecularly targeting drugs, trastuzumab, rituximab, imatinib, gefitinib, erlotinib, bevacizumab, cetuximab, panitumumab, bortezomib, sunitinib, sorafenib, crizotinib and regorafenib may be used. As an example of hormonal agents, dexamethasone, finasteride and tamoxifen may be used. As an example of biologics, Interferon α, β, and γ, and interleukin 2 may be used.

As an example of aphthalocyanine dyes, commercially available products, such as IRDye (registered trademark) 700DX, may be used. In the present invention, the NHS ester in IRDye (registered trademark) 700DX may be reacted with biotin modified dimers having amino groups to produce conjugates. Alternatively, other variations of IRDye (registered trademark) 700DX may be used, such variations are described in U.S. Pat. No. 7,005,518.

As a phthalocyanine dye, a dye represented by the following formula (21) can be used.

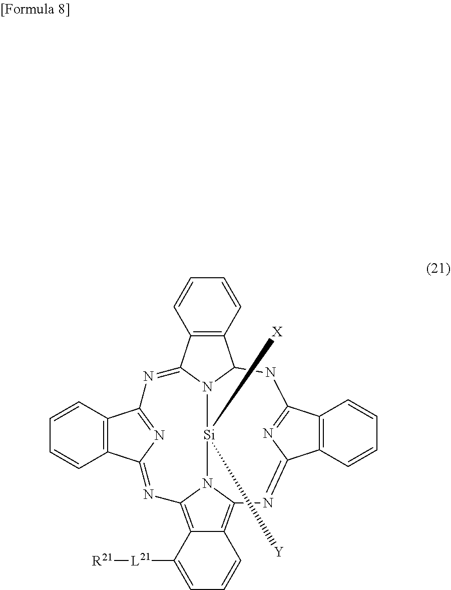

In the above formula (21), L²¹ represents a divalent linkage group, and R²¹ represents a functional group which can bind with other compounds.

X and Y each independently represents a hydrophilic group, —OH, a hydrogen atom, or a substituent. The substituent herein may be, but are not limited to, for example, a halogen atom (a fluorine atom), substituents containing carbon atoms (e.g. hydrocarbon groups), or substituents containing nitrogen atoms (e.g. amino groups).

In a specific example of X and Y,
(i) both X and Y may represent a hydrophilic group; or
(ii) either X or Y may represent a hydrophilic group and the other may represent —OH or a hydrogen atom; or
(iii) X and Y may represent —OH or a hydrogen atom.

The hydrophilic group represented by X and/or Y is not particularly limited, but for example, may be shown as follows.

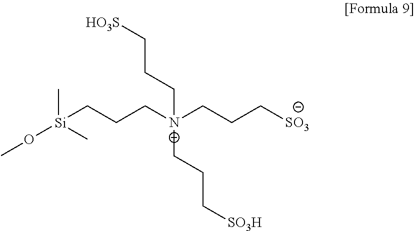

R²¹ preferably represents a functional group which can react and bind with carboxyl group, amine, or thiol groups on the biotin modified dimer. R²¹ may be preferably an activated ester, acyl halide, alkyl halide, amine which may suitably have a substituent, anhydride, carboxylic acid, carbodiimide, hydroxyl, iodoacetamide, isocyanate, isothiocyanate, maleimide, NHS ester, phosphoramidite, sulfonate, thiol, or thiocyanate, but not limited to these.

L²¹ represents a divalent linking group, for example, which may comprise any combination of ether, thioether, amine, ester, carbamate, urea, thiourea, oxy or amide bonds, or single, double, triple or aromatic carbon-carbon bonds; or phosphorus-oxygen, phosphorus-sulfur, nitrogen-nitrogen, nitrogen-oxygen or nitrogen-platinum bonds; or aromatic or heteroaromatic bonds. L²¹ is preferably a group consisting of a combination of groups selected from —CONH—, —NHCO—, —COO—, —OCO—, —CO—, —O—, and an alkylene group containing 1 to 10 carbon atoms.

L²¹-R²¹ may have phosphoramidite groups, NHS esters, activated carboxylic acids, thiocyanates, isothiocyanates, maleimides, and iodoacetamides.

L²¹ may contain —(CH₂)ₙ— groups, wherein n represents an integer of 1 to 10, preferably an integer of 1 to 4. -L²¹-R²¹, for example, may —O—(CH₂)₃—OC(O)—NH—(CH₂)₅—C(O)O—N-succinimidyl.

As a phthalocyanine dye, a dye represented by the following formula, which is disclosed in Japanese Unexamined Patent Application Publication No. JP-A-2021-54739 can be used.

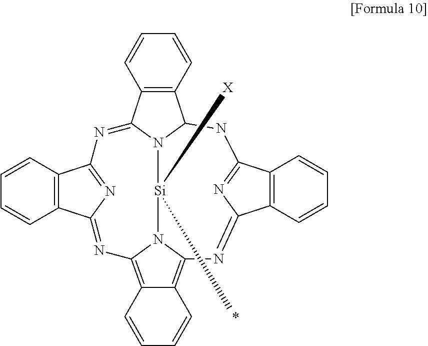

X represents a substituent having a hydrophilic group, cationic group, or amino group at the terminus thereof.
Asterisk represents a binding site with a linkage group for linking biotin modified dimer.

### <Photoimmunotherapy>

Photoimmunotherapy is a therapeutic method of using a photosensitizer and an irradiation light to destroy specific cells in a body. When a photosensitizer is exposed to a light with a specific wavelength, it generates cytotoxic reactive oxygen species capable of inducing apoptosis, necrosis, and/or autophagy to around cells. For example, Japanese Patent No. 6127045 discloses a method of killing cells, comprising: a step of allowing cells comprising a cell surface protein to come into contact with a therapeutically effective amount of one or more antibodies-IR700 molecules, wherein the antibodies specifically bind to the cell surface protein; a step of irradiating the cells with a light at a wavelength of 660 to 740 nm and at a dose of at least 1 Jcm⁻²; and a step of allowing the cells to come into contact with one or more therapeutic agents at approximately 0 to 8 hours after the irradiation, thereby killing the cells. JP Patent Publication (Kohyo) No. 2017-524659 A discloses a method of inducing cytotoxicity to a subject affected with a disease or a pathology, comprising: (a) administering to a subject, a therapeutically effective drug comprising a phthalocyanine dye such as IRDye (registered trademark) 700DX conjugated with a probe specifically binding to the cell of the subject; and (b) irradiating the cell with an appropriate excitation light in an amount effective for inducing cell death.

The fusion protein of the present invention and the conjugate of a biotin modified dimer and a phthalocyanine dye are administered to a subject, and the cells are then irradiated with an excitation light in an amount effective for suppression of cell proliferation or induction of cell death, so that the cell proliferation can be suppressed or the cell death can be induced, and thereby the subject can be treated.

Preferably, the fusion protein of the present invention and the conjugate of a biotin modified dimer and a phthalocyanine dye are administered to a subject, and the cells are then irradiated with an excitation light in an amount effective for suppression of cell proliferation or induction of cell death, so that the cell proliferation can be suppressed or the cell death can be induced, and thereby the subject can be treated.

It is preferable that the conjugate of a biotin modified dimer and a phthalocyanine dye and the fusion protein of the present invention are each administered in a therapeutically effective amount. Regarding each of the above-described conjugate and fusion protein, the therapeutically effective amount per 60 kg is at least 0.5 mg (mg/60 kg), at least 5 mg/60 kg, at least 10 mg/60 kg, at least 20 mg/60 kg, at least 30 mg/60 kg, or at least 50 mg/60 kg. For example, when it is intravenously administered, the applied dose is 1 mg/60 kg, 2 mg/60 kg, 5 mg/60 kg, 20 mg/60 kg, or 50 mg/60 kg, and it is, for example, 0.5 to 50 mg/60 kg. In another example, the therapeutically effective amount is at least 100 μg/kg, at least 500 μg/kg or at least 500 μg/kg, and it is, for example, at least 10 μg/kg. For example, when it is intratumorally or intraperitoneally administered, the dose is 100 μg/kg, 250 μg/kg, approximately 500 μg/kg, 750 μg/kg, or 1000 μg/kg, and it is, for example, 10 μg/kg to 1000 μg/kg. In one example, when it is administered in the form of a solution for local administration, the therapeutically effective amount is 10 μg/ml, 20 μg/ml, 30 μg/ml, 40 μg/ml, 50 μg/ml, 60 μg/ml, 70 μg/ml, 80 μg/ml, 90 μg/ml, 100 μg/ml or the like, or it is 20 μg/ml to 100 μg/ml, or it is at least 500 μg/ml, or at least 1 μg/ml.

The above-described dose can be administered once or divided doses over several administrations (2, 3, or 4 times, etc.), or as a single preparation.

The conjugate of a biotin modified dimer and a phthalocyanine dye and the fusion protein of the present invention can be each administered alone, or can also be administered in the presence of a pharmaceutically acceptable carrier, or can also be administered in the presence of other therapeutic agents (other anticancer agents, etc.).

The conjugate of a biotin modified dimer and a phthalocyanine dye and the fusion protein of the present invention can bind to target cells or target tissues, such as circulating tumor cells or solid tumor cells. Thereafter, the target cells or tissues are irradiated with a light, so that the above-described conjugate or complex can absorb the light and can damage or destroy the target cells or tissues.

In the photoimmunotherapy, the wavelength of the irradiation light is preferably 660 to 740 nm, and the irradiation light has a wavelength of, for example, 660 nm, 670 nm, 680 nm, 690 nm, 700 nm, 710 nm, 720 nm, 730 nm, or 740 nm. Light irradiation may be carried out using a device equipped with a near infrared (NIR) light emitting diode.

The light irradiation amount is at least 1 J/cm², for example, at least 4 J/cm², at least 10 J/cm², at least 15 J/cm², at least 20 J/cm², at least 50 J/cm², or at least 100 J/cm². It is, for example, 1 to 500 J/cm². Light irradiation may be carried out several times (e.g., 2, 3, 4, 5, 6, 7, 8, 9, or 10 times).

### <The Subject to be Treated>

The subject to be treated used herein includes humans and non-human animals. Examples of the subject may include humans and experimental animals such as mice. The subject is preferably affected with a disease regarding which suppression of cell proliferation or induction of cell death is desired. For example, the subject is affected with a cancer or a solid tumor.

Examples of the “cancer” may include carcinoma, lymphoma, blastoma, sarcoma, and leukemia or malignant lymphoma. Specific examples of the cancer may include squamous cell carcinoma (e.g., epithelial squamous cell carcinoma), lung cancer including small cell lung cancer, non-small cell lung cancer (“NSCLC”), pulmonary adenocarcinoma and pulmonary squamous cell carcinoma, peritoneal cancer, hepatocarcinoma, corpus ventriculi or stomach cancer, including digestive cancer, pancreatic cancer, glioblastoma, cervical cancer, ovarian cancer, liver cancer, bladder cancer, hepatocellular cancer, breast cancer, colon cancer, rectal cancer, colorectal cancer, endometrial membrane cancer or endometrial carcinoma, salivary gland carcinoma, kidney or renal region cancer, prostate cancer, vulvar cancer, thyroid cancer, hepatocellular carcinoma, anal carcinoma, penile carcinoma, and head and neck cancer.

The solid tumor means a benign or malignant, abnormal cell mass that generally does not contain a capsule. Examples of the solid tumor may include glioma, astrocytoma, medulloblastoma, craniopharyngioma, ependymoma, pineal gland tumor, hemangioblastoma, acoustic neuroma, oligodendrocyte, meningioma, melanoma, neuroblastoma, and retinoblastoma.

Examples of the administration method to the subject may include, but are not limited to, a local route, an injection (a subcutaneous injection, an intramuscular injection, an intradermal injection, an intraperitoneal injection, an intratumoral injection, an intravenous injection, etc.), an oral route, an ocular route, a sublingual route, a rectal route, a percutaneous route, an intranasal route, a vaginal route, and an inhalation route.

The present invention will be more specifically described in the following examples. However, these examples are not intended to limit the scope of the present invention.

### Examples

### <Preparation of Expression Vector>

Cupid molecules (SEQ ID NO: 1) (International Publication WO2015/125820) were fused with molecules recognizing the HER2 antigen, a single-domain antibody (sdAb, VHH, nanobody) (non-patent document: I. Vaneycken, et al. FASEB J 2011, 25, 2433) (SEQ ID NO: 2), and a protein having binding ability to both Psyche molecules (the above “Psyche J”) and Her2/ErbB2 (hereinafter this protein is referred to as “Anti-HER2 VHH-Cupid”) was constructed (SEQ ID NO: 3). The theoretical molecular weight is approximately 27.5 kDa for the monomer and 110 kDa for the tetramer. The gene sequence of Anti-HER2 VHH-Cupid was synthesized as an artificial gene (SEQ ID NO: 4) by Eurofins.

SEQ ID NO: 1: amino acid sequence of Cupid
AEAGITGTWSDQLGDTFIVTAGADGALTGTYENAVGGAESRYVLTGRYD
SAPATDGSGTALGWTVAWKNNSKNAHSATTWSGQYVGGADAKINTQWLL
TSGTTNANAWKSTLVGHDTFTKVKPSAASHHHHHH
SEQ ID NO: 2:
MQVQLQESGGGSVQAGGSLKLTCAASGYIFNSCGMGWYRQSPGRERELV
SRISGDGDTWHKESVKGRFTISQDNVKKTLYLQMNSLKPEDTAVYFCAV
CYNLETYWGQGTQVTVSS
SEQ ID NO: 3:
MQVQLQESGGGSVQAGGSLKLTCAASGYIFNSCGMGWYRQSPGRERELV
SRISGDGDTWHKESVKGRFTISQDNVKKTLYLQMNSLKPEDTAVYFCAV
CYNLETYWGQGTQVTVSSGGGGSGGGGSGGGGSAEAGITGTWSDQLGDT
FIVTAGADGALTGTYENAVGGAESRYVLTGRYDSAPATDGSGTALGWTV
AWKNNSKNAHSATTWSGQYVGGADAKINTQWLLTSGTTNANAWKSTLVG
HDTFTKVKPSAASHHHHHH*
SEQ ID NO: 4:
CAGGTTCAGCTGCAAGAAAGCGGTGGTGGTAGCGTTCAGGCAGGCGGTA
GCCTGAAACTGACCTGTGCAGCAAGCGGTTATATCTTTAATAGCTGTGG
TATGGGTTGGTATCGTCAGAGTCCGGGTCGTGAACGTGAACTGGTTAGC
CGTATTAGCGGTGATGGTGATACCTGGCATAAAGAAAGCGTTAAAGGTC
GTTTTACCATCAGCCAGGATAATGTTAAAAAGACCCTGTACCTGCAGAT
GAATAGTCTGAAACCGGAAGATACCGCAGTGTATTTTTGTGCCGTTTGC
TATAATCTGGAAACCTATTGGGGTCAGGGCACCCAGGTTACCGTTAGCA
GCGGAGGCGGAGGGTCTGGAGGTGGCGGTTCAGGTGGCGGTGGCAGTGC
GGAAGCCGGTATTACCGGGACCTGGTCCGATCAACTCGGCGACACGTTT
ATCGTGACAGCAGGCGCGGATGGCGCGCTGACAGGGACGTACGAGAATG
CGGTAGGCGGTGCGGAAAGCCGCTATGTGTTGACCGGTCGTTACGATTC
GGCTCCGGCTACGGATGGTTCCGGTACAGCCTTAGGGTGGACCGTTGCG
TGGAAGAACAACTCGAAGAATGCCCACAGTGCTACCACTTGGTCAGGCC
AGTATGTTGGCGGGGCGGATGCGAAAATCAACACTCAGTGGCTTCTGAC
CAGCGGAACCACGAATGCCAATGCGTGGAAATCCACGCTGGTCGGTCAT
GACACCTTTACCAAAGTCAAACCGAGTGCAGCATCGCATCATCACCACC
ATCAT

An expression vector was prepared using pET45b according to a common method. Specifically, using a primer set (Rv: CATggtatatctccttcttaaagttaaac (SEQ ID NO: 5), Fw: TAACGCAGCTTAATTAACCTAGGCTG (SEQ ID NO: 6)), the vector was linearized by a PCR reaction. The sequence prepared by the artificial gene synthesis was amplified by a PCR reaction using a primer set (Fw: AGGAGATATACCATGCAGGTTCAGCTGCAAGAAAGCGGTG (SEQ ID NO: 7); Rv: AATTAAGCTGCGTTAATGATGGTGGTGATGATGCGATGCTG (SEQ ID NO: 8)). Bands were cut out of both of the PCR reaction products according to agarose gel electrophoresis, and were then purified. Using In-Fusion HD Cloning Kit (TaKaRa Bio), the purified vector and the insert were subjected to a ligation reaction and cloning in accordance with the dosage and administration described in the instruction manual.

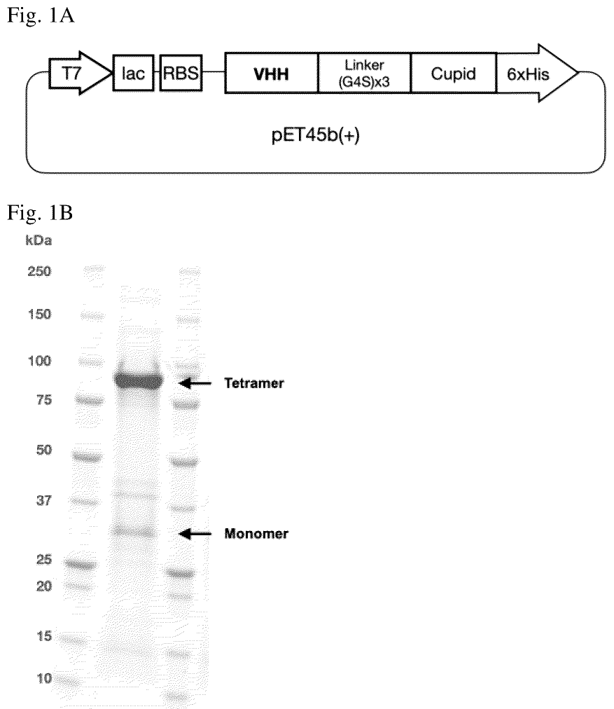

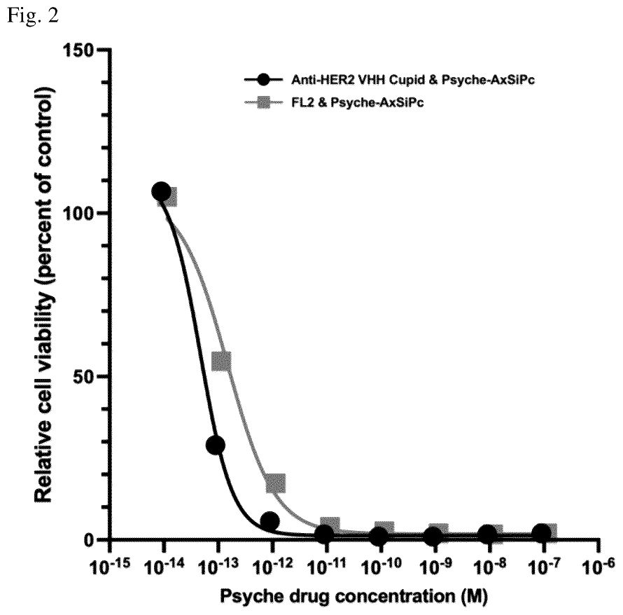

The map of the expression vector is shown in FIG. 1A.

A plasmid vector, which was confirmed after cloning by sequence analysis that a gene of interest had been incorporated therein, was introduced into competent cells BL21(DE3) (ECOS Competent E. coli BL21(DE3), Nippon Gene Co., Ltd.), so that the cells were transformed. A culture medium prepared by culturing the cells in 100 mL of 2×YT medium overnight was inoculated into 1 L of a culture solution, and the obtained mixture was then cultured at 37° C. When the OD value at 600 nm became 0.5 to 0.8, IPTG was added to the culture to a final concentration of 0.5 mM, and the thus obtained mixture was then cultured at 37° C. for 4 hours. Thereafter, the resulting cells were recovered by centrifugation (7500×g, 20 min at 4° C.).

### <Preparation of IBs>

Then, the method of recovering IBs is described below. After suspended in Lysis buffer (20 mM Tris-HCl, 2 mM MgCl₂, 10 μg/mL Lysozyme, from Egg White, 25 units/mL Benzonase, pH8.0), the mixture was incubated at room temperature for 10 minutes, and an insoluble fraction (inclusion body; hereinafter referred to as “IB”) was separated from a soluble fraction by centrifugation. Thereafter, the IBs were recovered. Subsequently, the recovered IBs were resuspended in a 10-fold diluted Lysis buffer (without addition of Benzonase), and was centrifuged. The supernatant was discarded, and the washing of IBs was repeated three times. After completion of the washing three times, the IBs recovered by centrifugation were resuspended in ultrapure water (Milli Q). Thereafter, the resuspension was dispensed in an amount of 1 mL each into 1.5-mL tubes, and was then cryopreserved at −80° C.

### <The Method of Denaturing and Refolding IBs>

Then, denaturation and refolding of IBs are described below. A denaturing buffer (6 M guanidium HCl, 150 mM NaCl, 50 mM Tris-HCl, and 375 μM 2-mercaptoethanol, pH 8.0) was added to the IBs, and the IBs were then dissolved in the buffer by pipetting. Thereafter, while stirring the obtained solution using a rotator, the solution was incubated at 25° C. for 30 minutes, and was then centrifuged (15,000×g, 10 min at 4° C.) to recover a supernatant. The recovered supernatant was adjusted with the denaturing buffer, so the protein concentration (OD 280 nm) in the supernatant became 30 to 50 mg/mL. Thereafter, the denatured protein solution was 25-fold diluted with refolding buffer A (0.4 M arginine HCl, 150 mM NaCl, 50 mM Tris-HCl, 375 μM Glutathione, Oxidized Form, pH 8.0). Thereafter, the obtained solution was incubated at 4° C. for 1 to 2 hours and then was 2-fold diluted with refolding buffer B (2 M guanidium HCl, 0.4 M arginine HCl, 150 mM NaCl, 50 mM Tris-HCl, 375 μM Glutathione, Oxidized Form, pH 8.0). Thereafter, the obtained solution was incubated at 4° C. for 16 to 48 hours.

Then, the sample was put into dialysis tube and dialyzed with 0.1M sodium phosphate, 0.4M arginine HCl, pH5.5. After dialysis, aggregates were removed by centrifugation (15,000×g, 10 min at 4° C.) and the supernatant was recovered. The recovered supernatant was concentrated by using VIVASPIN Turbo 15 MWCO 30,000 (Sartorius AG). The electrophoretogram of the refolded sample is shown in FIG. 1B.

FL2 (Z_{HER2:342}-Cupid-His) was used as a control for comparison, which was prepared according to the method described in International Publication WO2022/203000, International Publication WO2023/008515, and non-patent document (Yamatsugu et al. Protein Expr. Purif. 2022, 192, 106043. DOI: 10.1016/j.pep.2021.106043). FL2 was described as ZHER2:342-Cupid-His in Yamatsugu et al. Protein Expr. Purif. 2022, 192, 106043.

### <Cytotoxic Activity Confirmed Using Anti-HER2 VHH-Cupid and Photosensitizer Psyche>

Her2-positive cells (KPL-4) were seeded on a 96-well plate to result in a cell count of 1×10⁴ cells/well and a culture solution amount of 50 μL/well and were then cultured overnight. Anti-HER2 VHH-Cupid and FL2 were mixed with the photosensitizer Psyche (prepared with reference to Yamatsugu et al. Protein Expr. Purif. 2022, 192, 106043. DOI: 10.1016/j.pep.2021.106043) at a molar ratio of 1:2 (hereinafter referred to as a “complex”), and 2-fold dilution series, with seven concentrations, were prepared from 20 μg/mL. After the dilution series had been prepared, 50 μL of the complex was each added to the cells, and 24 hours later, the cells were irradiated with an LED emitting a 690-nm light at 100 J/cm² from the bottom surface of the culture plate. Thereafter, the cells were cultured for 24 hours, and a comparison was then made in terms of the number of living cells, using Cell Counting Kit-8 (DOJINDO LABORATORIES). A reagent was added in accordance with the dosage and administration described in the instruction manual, and the resulting cells were then incubated at 37° C. in a CO₂ incubator for 1.5 hours. After that, the absorbance at 450 nm was measured, and the mean value was then calculated, followed by background correction. Thereafter, the control was set at 100%, and the cell proliferation percentage to the control under individual conditions was calculated. The results are shown in FIG. 2. It was confirmed that the complex of the Anti-HER2 VHH-Cupid and the photosensitizer Psyche compound had concentration-dependent cytotoxicity. In addition, IC₅₀ were calculated from the graphs in FIG. 2 by using the software GrafPad Prism (Dotmatics), and the comparison of IC₅₀ is shown in Table 1. It was confirmed that the value of IC₅₀ was smaller with Anti-HER2 VHH-Cupid as a vector than with FL2. This indicates that Anti-HER2 VHH-Cupid has stronger cytotoxic activity than FL2.

### The Photosensitizer Psyche

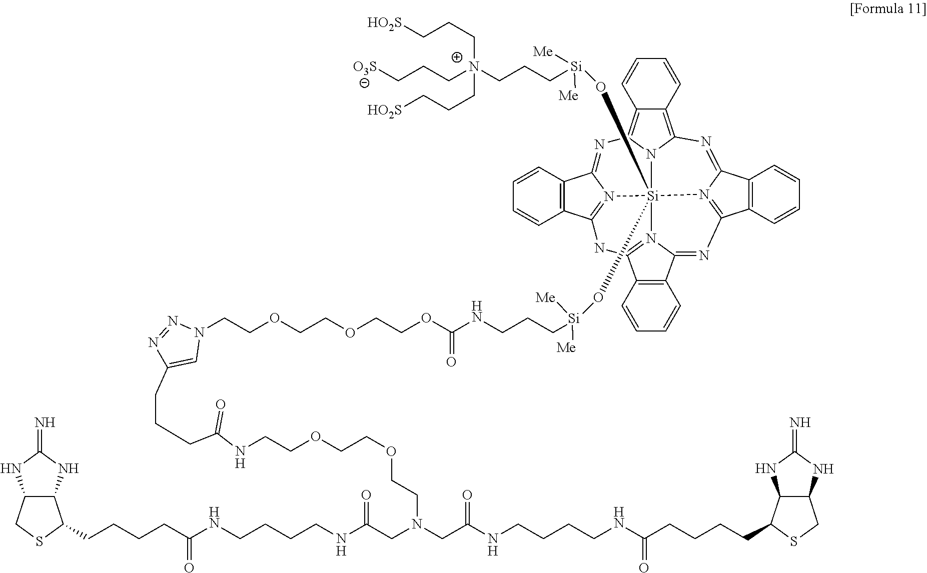

### <Confirmation of Cytotoxic Activity Using Anti-HER2 VHH-Cupid and Anti-Cancer Drug Psyche (Psyche-Duocarmycin)>

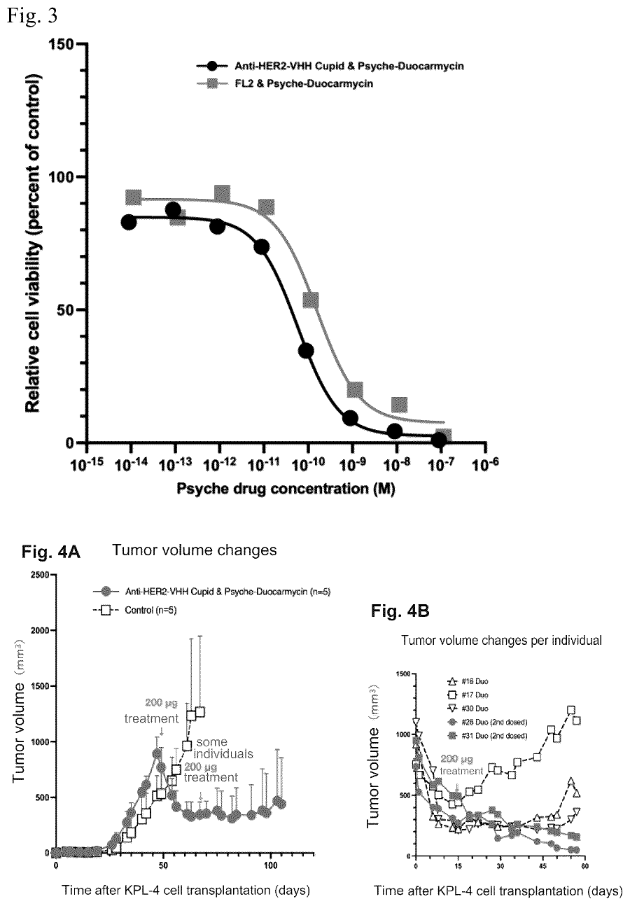

Her2-positive cells (KPL-4) were seeded on a 96-well plate to result in a cell count of 1×10³ cells/well and a culture solution amount of 50 μL/well and were then cultured overnight. Anti-HER2 VHH-Cupid and FL were mixed with the anti-cancer drug Psyche (Psyche-Duocarmycin: which was prepared with reference to International Publication WO2021/215534) at a molar ratio of 1:2 (hereinafter referred to as a “complex”), and 2-fold dilution series, with seven concentrations, were prepared from 20 μg/mL. After the dilution series had been prepared, 50μ of the complex was each added to the cells, and the cells are cultured for 6 to 7 days. A comparison was then made in terms of the number of living cells, using Cell Counting Kit-8 (DOJINDO LABORATORIES). A reagent was added in accordance with the dosage and administration described in the instruction manual, and the resulting cells were then incubated at 37° C. in a CO₂ incubator for 1.5 hours. After that, the absorbance at 450 nm was measured, and the mean value was then calculated, followed by background correction. Thereafter, the control was set at 100%, and the cell proliferation percentage to the control under individual conditions was calculated. The results are shown in FIG. 3. It was confirmed that the complex of the Anti-HER2 VHH-Cupid and Psyche-Duocarmycin compound had concentration-dependent cytotoxicity. In addition, IC₅₀ were calculated from the graphs in FIG. 3 by using the software GrafPad Prism (Dotmatics), and the comparison of IC₅₀ is shown in Table 1. It was confirmed that the value of IC₅₀ was smaller with Anti-HER2 VHH-Cupid as a vector than with FL2. This indicates that Anti-HER2 VHH-Cupid has stronger cytotoxic activity than FL2.

TABLE 1
The comparison of IC₅₀

| | IC₅₀ by Psyche | IC₅₀ by Psyche |
|---|---|---|
| Vector | Ax-SiPc [M] | Duocarmycin [M] |
| Anti-HER2 VHH-Cupid | 4.72E−14 | 5.77E−11 |
| FL2 | 1.48E−13 | 1.59E−10 |

### <In Vivo Experiment in Xenograft Model Mouse>

KPL-4 cells cultured in DMEM containing 10% FBS were transplanted into the subcutis of nude mice (5- to 10-week-old) in an amount of 7.5×10⁶ cells/body. After completion of the transplantation, mice with a tumor mass volume of 300 to 1000 mm³ were administered, via the caudal vein, with the complex of the Anti-HER2 VHH-Cupid and Psyche-Duocarmycin at a dose of 200 μg/body. The volume of the tumor mass was obtained by measuring the long diameter and short diameter of the tumor mass with Vernier calipers, and then by applying the formula: (short diameter)²×long diameter×½. The results are shown in FIG. 4A. By the comparison of the treatment group (n=5) and control group (n=5), it was confirmed that a tumor proliferation was suppressed in the treatment group with the complex of Anti-HER2 VHH-Cupid and Psyche-Duocarmycin. Moreover, #26 and #31 mice were received an additional treatment at 14 days after the initial treatment. The tumor proliferation was suppressed in the two mice administered additionally, as shown in FIG. 4B. In contrast, the tendency to tumor proliferation was observed in three mice without any additional treatment.

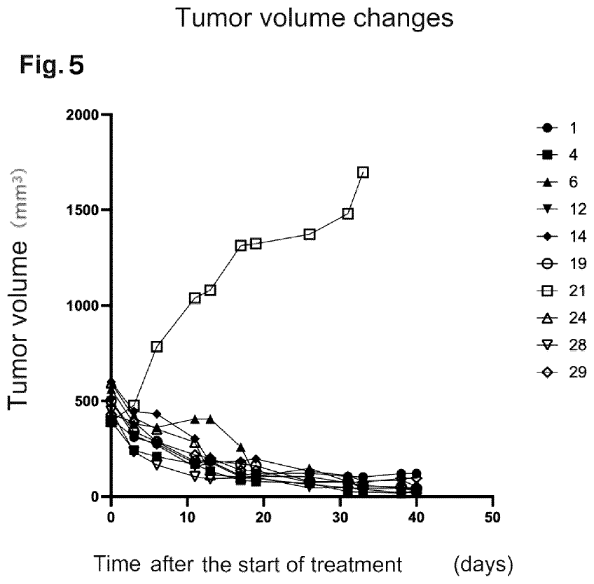

Moreover, new ten mice were administered with the complex of Anti-HER2 VHH-Cupid and Psyche-Duocarmycin at a dose of 200 μg/body. The results are shown in FIG. 5. One mouse of these mice probably failed to be administered via the caudal vein. It was confirmed that the other nine mice showed the suppression of tumor proliferation, similarly to FIG. 4, with good reproducibility.

### <Expression of Anti-HER2 VHH-Cupid>

A plasmid vector, where Anti-HER2 VHH-Cupid gene had been incorporated, was introduced into competent cells BL21(DE3), so that the cells were transformed. A culture medium prepared by culturing the cells in 100 mL of 2×YT medium at 37° C. overnight was inoculated into 1 L of a culture solution, and the obtained mixture was then cultured at 37° C. When the OD value at 600 nm became 0.8, IPTG was added to the culture to a final concentration of 1.0 mM, and the thus obtained mixture was then cultured at 37° C. for 4 hours. Thereafter, the resulting cells were recovered by centrifugation (7500×g, 10 min at 4° C.).

### <Preparation of IBs>

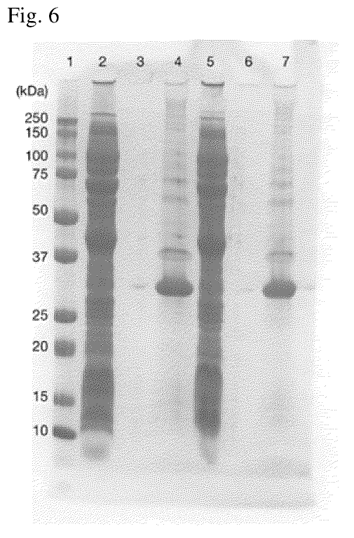

Then, the method of recovering IBs is described below. After suspended in Lysis buffer (20 mM Tris-HCl, 2 mM MgCl₂, 10 μg/mL Lysozyme from Egg White, 2 units/μL Benzonase, pH 8.0), the mixture was sonicated to remove nucleic acid. Thereafter, an insoluble fraction was separated from a soluble fraction by centrifugation to recover IBs in the insoluble fraction. Subsequently, the recovered IBs were resuspended in a 10-fold diluted Lysis buffer (without addition of Benzonase), and then they were centrifuged. The supernatant was discarded, and the washing of the IBs was repeated three times. After completion of the washing three times, the IBs recovered by centrifugation were resuspended in ultrapure water (Milli Q). Thereafter, the resuspension was dispensed in an amount of 1 mL each into 1.5-mL tubes, and was then cryopreserved at −80° C. After induction by IPTG, the lysate was analyzed by non-reducing electrophoresis and the result image is shown in FIG. 6. The soluble fractions of the cell lysate are shown in lanes 2, 5, and the insoluble fractions of the cell lysate are shown in lanes 4, 7. IBs are comprised primarily of anti-HER2 VHH-Cupid, with only a few additional bands representing host cell proteins.

### <The Method of Denaturing and Refolding IBs>

Then, denaturation and refolding of IBs are described below. A solubilization buffer (6 M guanidium Cl, 150 mM NaCl, 50 mM Tris-HCl, 375 μM 2-mercaptoethanol, pH 8.0) was added to IBs, and the IB was then dissolved in the buffer by pipetting. Thereafter, while stirring the obtained solution on a shaker, the solution was incubated at 25° C. for 1 hour, and was then centrifuged (12,000×g, 15 min at 4° C.) to recover a supernatant. Thereafter, the recovered supernatant was 25-fold diluted with refolding buffer A (2 M guanidium Cl, 0.4 M arginine HCl, 150 mM NaCl, 50 mM Tris-HCl, 375 μM Glutathione Oxidized Form, pH 8.0). Thereafter, the obtained solution was incubated at 4° C. for 1 hour and then was 2-fold diluted with refolding buffer B (0.4 M arginine HCl, 150 mM NaCl, 50 mM Tris-HCl, 375 μM Glutathione Oxidized Form, pH 8.0). Thereafter, the obtained solution was incubated at 4° C. for 24 to 48 hours.

### <Study for pH Conditions of Dialysis Buffer Optimal for Tetramer Formation of Anti-HER2 VHH-Cupid>

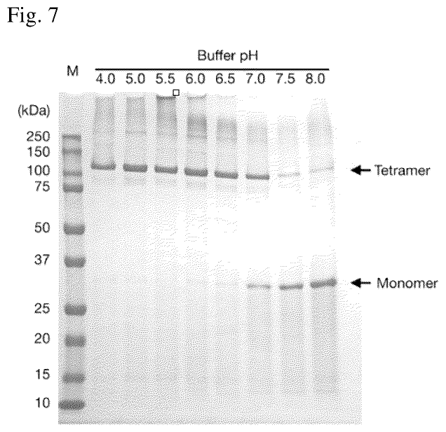

Then, the sample was put into dialysis tube and dialyzed with buffers (50 mM sodium phosphate, 400 mM arginine HCl, pH 4.0˜8.0). 100 μL of the sample after refolding was dialyzed at 25° C. for 1 hour using 30 mL of buffer and the Mini Dialysis Kit 1 kD (Cytiva). Thereafter, dialysis was performed at 4° C. for 18 hours with 30 mL of fresh buffer, followed by overnight dialysis at 4° C. with 30 mL of fresh buffer. The post-dialysis samples from each pH condition were analyzed by non-reducing electrophoresis and the result image was shown in FIG. 7. The refolded sample formed tetramers in pH-dependent manner. Moreover, relative percentages of monomers and tetramers in samples from each pH condition were calculated by densitometric analysis of each band, and the results were showed in Table 2. As shown in FIG. 7 and Table 2, the monomers were equal to or less than 8% and the tetramers were equal to or more than 92% in pH of 6.0 or less, whereas the percentage of monomers increased at a pH of 6.5 or more. At pH 8.0, the same as the pH of refolding buffer B, 81% of the monomers and 19% of the tetramers were present. The theoretical isoelectric point (pI) of Anti-HER2 VHH-Cupid was calculated as approximately 6.7. The theoretical pIs of Anti-HER2 VHH and Cupid moieties were approximately 7.6 and 6.5, respectively. Therefore, it is preferable that a buffer for tetramer formation should be pH 6.0 or less to avoid aggregation induced by the Cupid moiety. Moreover, a refolded Anti-HER2 VHH-Cupid, which was dialyzed with a refolding buffer B to remove guanidine hydrochloride, was also tested by non-reducing electrophoresis. The result image showed that almost all of the Anti-HER2 VHH-Cupid were monomeric.

TABLE 2
The pH condition of dialysis buffer and the population
ratio of Anti-HER2 VHH-Cupid monomers and tetramers

| Buffer pH | Monomer band (%) | Tetramer band (%) |
|---|---|---|
| pH 4.0 | 4 | 96 |
| pH 5.0 | 6 | 94 |
| pH 5.5 | 8 | 92 |
| PH 6.0 | 8 | 92 |
| pH 6.5 | 13 | 87 |
| pH 7.0 | 35 | 65 |
| pH 7.5 | 77 | 23 |
| pH 8.0 | 81 | 19 |

### <Recovering of Tetrameric Anti-HER2 VHH-Cupid by Size Exclusion Chromatography>

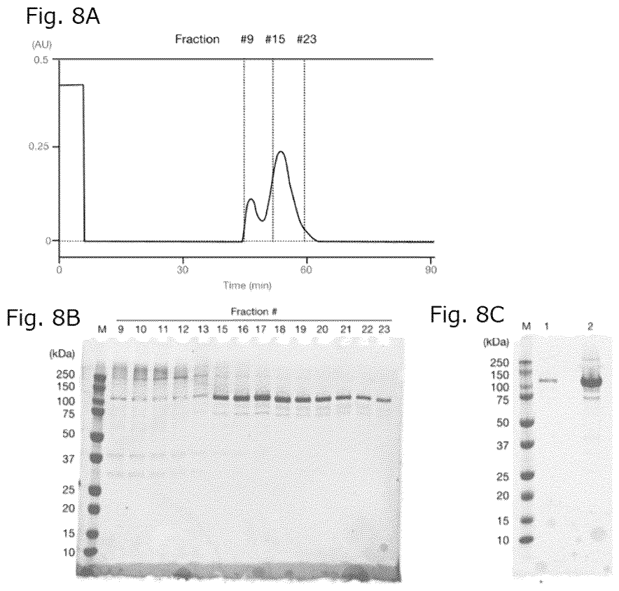

Then, the refolded Anti-HER2 VHH-Cupid was dialyzed against the refolding buffer C (50 mM sodium phosphate, 400 mM arginine HCl, pH 5.0) and centrifuged (15,000×g, 10 min, at 4° C.) to remove aggregates. The supernatant was recovered and concentrated to >10 mg/mL using Vivaspin Turbo 15 PES, 50,000 MWCO (Sartorius). The concentrated sample was filtered with a 0.22 μm Millex syringe filter (Merck Millipore), and the resulting sample was loaded onto a gel filtration column (HiLoad 16/600 Superdex 75 μg; Cytiva). Size exclusion chromatography was performed using size exclusion chromatography (SEC) buffer (20 mM sodium phosphate, 0.2 M arginine HCl, pH 5.5). The result is shown in FIG. 8. As shown in FIG. 8A, the chromatogram exhibited two peaks. The fractions from each retention time were analyzed by non-reducing electrophoresis, and the result image are shown in FIG. 8B. This image revealed that the first peak contained monomers and the second peak contained mainly tetramers. Fractions #15-23 were pooled and concentrated for further in vitro and in vivo experiments. The concentrated tetrameric Anti-HER2 VHH-Cupid was confirmed by non-reducing electrophoresis, and the result image was shown in FIG. 8C. The concentrated Anti-HER2 VHH-Cupid tetramers were cryopreserved at −80° C.

### <Confirmation of Interaction Between Anti-HER2 VHH-Cupid and Anti-Cancer Drug Psyche (Psyche-Duocarmycin)>

The surface plasmon resonance (SPR) analysis was performed using a Biacore T200 (Cytiva). The anti-histidine antibody was fixed to a S Sensor Chip CM5 (Cytiva) to capture the Anti-HER2 VHH-Cupid protein using a His Capture Kit (Cytiva). Subsequently, 2-fold dilution series, with six concentrations (from 10 nM to 0.3125 nM), were prepared from 10 nM Psyche-Duocarmycin and used as analytes. The sensorgrams of Anti-HER2 VHH-Cupid and Psyche-Duocarmycin are shown in FIG. 9. The association and dissociation rate constants were evaluated using Biacore T200 Evaluation Software version 3 (Cytiva) by bivalent analyte model, and K_{D} values were calculated. The association rate constant (Ka₁) was 1.336×10⁴ [l/Ms], and the dissociation rate constant (Kd₁) was 1.184×10⁻⁴ [l/s]. The K_{D} [M] value was calculated by applying the formula: Kd₁/Ka₁ and was 8.86×10⁻¹⁰ [M.] These results suggest a stable non-covalent bond between Anti-HER2 VHH-Cupid and Psyche-Duocarmycin is formed. Therefore, it was confirmed that Anti-HER2 VHH-Cupid had binding activity to Psyche-Duocarmycin. However, the interactions are well characterized as avidity rather than affinity, because they do not involve one-to-one binding.

### <Confirmation of Interaction Between Anti-HER2 VHH-Cupid and HER2 Extracellular Domain (HER2 ECD)>

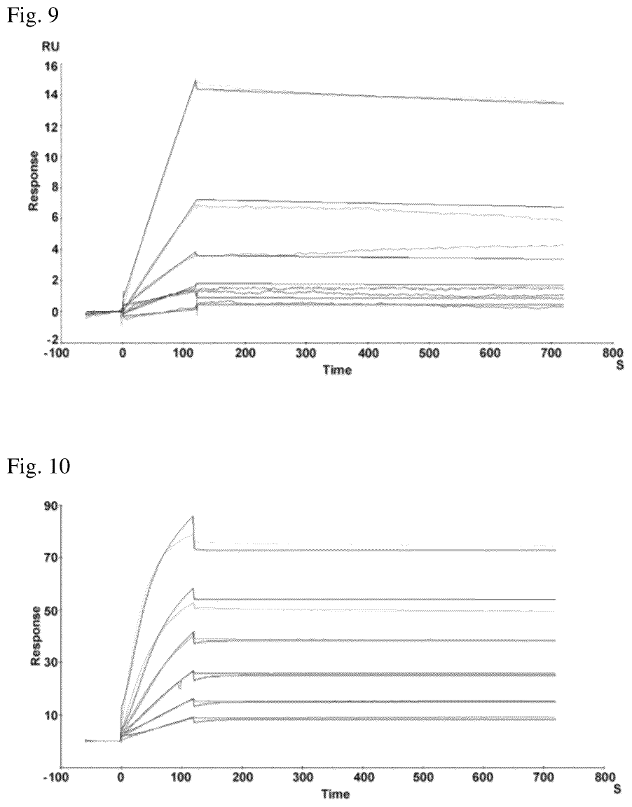

Likewise, the interaction between Anti-HER2 VHH-Cupid and HER2 extracellular domain (HER2 ECD) was confirmed using SPR analysis. An anti-human Fc antibody was fixed on the sensor chip. Recombinant human HER2 Fc chimera protein (R&D Systems) was captured via the Fc domain on the chip, using a Human Antibody Capture Kit (Cytiva). 2-fold dilution series, with six concentrations (from 10 nM to 0.3125 nM), were prepared from 10 nM Anti-HER2 VHH-Cupid and used as analytes. The sensorgrams of HER2 ECD and Anti-HER2 VHH-Cupid are shown in FIG. 10. The association rate constant (Ka₁) was 5.388×10⁶ [l/Ms], and the dissociation rate constant (Kd₁) was 7.732×10⁻⁶ [l/s]. The K_{D} [M] value was 1.435×10⁻¹² [M]. These results suggest that the Anti-HER2 VHH-Cupid and HER2 ECD interaction slowly dissociates and that a stable non-covalent bond between Anti-HER2 VHH Cupid and HER2 ECD is formed. Therefore, it was confirmed that Anti-HER2 VHH-Cupid had binding activity to HER2 ECD. However, the interactions are well characterized as avidity rather than affinity, because they do not involve one-to-one binding.

### <Confirmation of Internalization Activity of Anti-HER2 VHH-Cupid and Fluorochrome Psyche (Psyche-FITC)>

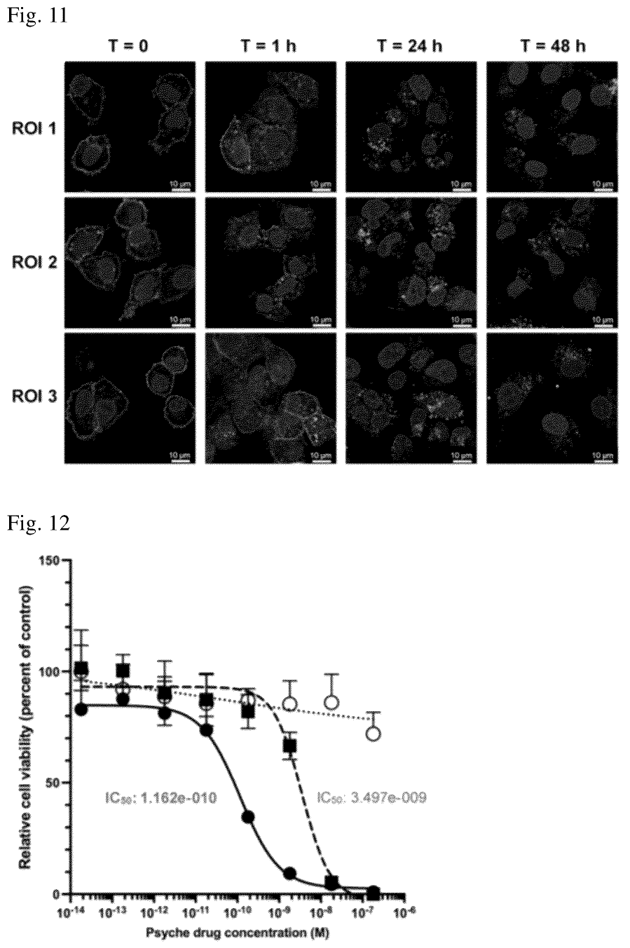

HER2-positive cells (KPL-4) were seeded at a 5.0×10⁴ cells/well density in 35 mm glass-bottom dishes (Iwaki). The cells were washed twice with prewarmed serum starvation medium and incubated at 37° C. for 1 hour. The cells were placed on ice in a 4° C. cold room for 15 min. Anti-HER2 VHH-Cupid and fluorochrome Psyche (Psyche-FITC) were mixed in a molar ratio of 1:2 in the dark on ice for 10 min to form a complex (hereinafter referred to as a “complex”). The medium was replaced with an ice-cold (2-4° C.) starvation medium containing 10 μg/mL of the complex. The cells were then incubated on ice at 4° C. for 30 min to label the cell surface with HER2 and washed twice with an ice-cold starvation medium. The cells were cultured at 37° C. for 0, 1, 24 and 48 hours, respectively. The cells were washed with 2 mL of ice-cold stripping buffer (pH 4.6 citrate buffer) at 4° C. for 2 min on ice and then briefly washed with ice-cold phosphate-buffered saline (PBS) to remove non-specifically bound proteins from the cell surface. Thereafter, the cells were fixed with a fixation buffer (4% paraformaldehyde) on ice for 4 min, rinsed with PBS, counterstained with 4′,6-Diamidino-2-phenylindole dihydrochloride solution (DAPI), and re-rinsed with PBS. The cells were detected, using the excitation wavelength at 408 and 488 nm, with the emission wavelength from 500 to 535 nm. Fluorescence was observed using a 63×1.40 NA oil immersion objective lens (HC PL APO 63×/1.40 Oil CS2, Leica). Lightning Mode (Leica Microsystems) was used to generate deconvolved images. Microscopic acquisitions were controlled using LAS X (v. 4.2.1) software (Leica). The results are shown in FIG. 11. Fluorescence signals were observed on the surface of the cell membrane, but as time progressed, they were observed as dots in the cytoplasm. These results showed that the complex of Anti-HER2 VHH-Cupid and Psyche-FITC had internalization activity.

### <Confirmation of Cytotoxic Activity Using Anti-HER2 VHH-Cupid and Anti-Cancer Drug Psyche (Psyche-Duocarmycin)>

Her2-positive cells (KPL-4) were seeded on a 96-well plate to result in a cell count of 1×10³ cells/well and a culture solution amount of 50 μL/well and were then cultured overnight. Anti-HER2 VHH-Cupid were mixed with the anti-cancer drug Psyche (Psyche-Duocarmycin) at a molar ratio of 1:2 (hereinafter referred to as a “complex”), and 10-fold dilution series, with seven concentrations, were prepared from 20 μg/mL. After the dilution series had been prepared, 50 μL of the complex, duocarmycin-Psyche alone, or Anti-HER2 VHH-Cupid alone were added to the cells respectively, and the cells were cultured at 37° C. for 6 days. Subsequently, a comparison was then made in terms of the number of living cells, using Cell Counting Kit-8 (DOJINDO LABORATORIES). 10 μL of CCK-8 reagent was added per well, in accordance with the dosage and administration described in the instruction manual, and the resulting cells were then incubated at 37° C. in a CO₂ incubator for 1.5 hours. After that, the absorbance at 450 nm was measured, and the mean value was then calculated, followed by background correction. Thereafter, the control was set at 100%, and the cell proliferation percentage to the control under individual conditions was calculated. The results are shown in FIG. 12. It was confirmed that the complex of Anti-HER2 VHH-Cupid and Psyche-Duocarmycin compound had concentration-dependent cytotoxicity. In addition, IC₅₀ were calculated from the graph in FIG. 12 by using the software GrafPad Prism (Dotmatics). The IC₅₀ value of the complex and Psyche-Duocarmycin were 0.116 nM and 3.49 nM, respectively. Thus, this indicates that the complex of Anti-HER2 VHH-Cupid and Psyche-Duocarmycin had 30-fold higher efficacy in cytotoxic activity than duocarmycin alone (i.e. the payload alone), when they were added to the cells.

### <In Vivo Experiment for Tumor Proliferation Suppressing Effect by the Interval Between Repeated Administrations in Xenograft Model Mouse>

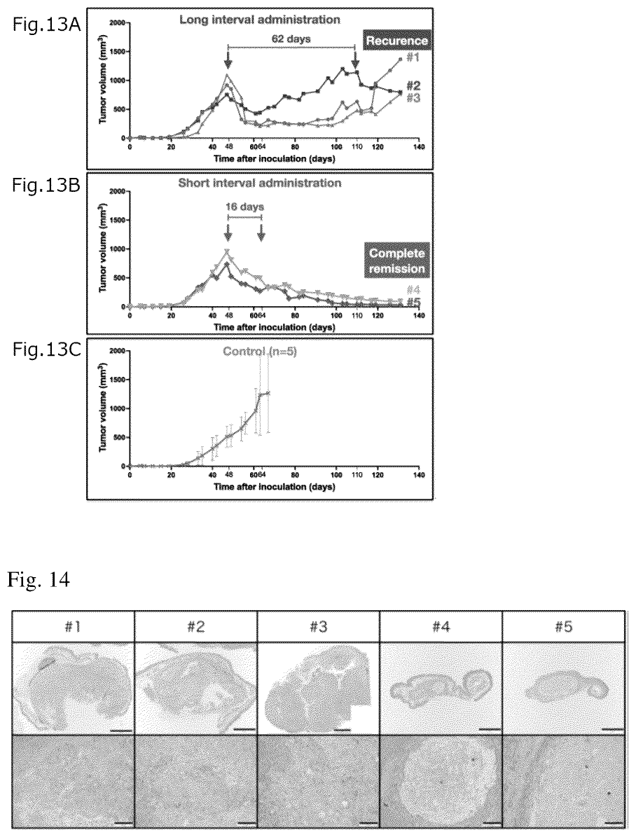

KPL-4 cells cultured in DMEM containing 10% FBS were subcutaneously transplanted into the thigh of ten nude mice (BALB/cSlc-nu/nu, 5 to 10-week-old, Sankyo Lab Service) in an amount of 7.5×10⁶ cells/body. The volume of the tumor mass was obtained by measuring the length and width of the tumor mass with calipers, and then by applying the formula: 0.5×length×width². Anti-HER2 VHH-Cupid were mixed with the anti-cancer drug Psyche (Psyche-Duocarmycin) at a molar ratio of 1:2 to prepare the complex of Anti-HER2 VHH-Cupid and Psyche-Duocarmycin (hereinafter referred to as a “complex”). The complex was diluted with PBS to a concentration of 1.33 mg/mL, thereafter five mice were administered, via the caudal vein, with the complex at a dose of 200 μg/body. In addition, an additional treatment was carried out in two different intervals, in a long-interval and in a short-interval after the initial treatment, at the same dose of the initial treatment. In the long-interval group, three mice were additionally administered 62 days after initial treatment. In the short-interval group, two mice were additionally administered 16 days after initial treatment. The remain five mice were observed without any treatment as a control group. The results of the long-interval group, the short-interval group, and the control group were respectively shown in FIG. 13A, FIG. 13B, and FIG. 13C. By the comparison of the treatment group (n=5) and the control group (n=5), it was confirmed that a tumor proliferation was suppressed in the treatment group with the complex immediately after the initial treatment. In the long-interval group (n=3), the tumor size began to increase again approximately two weeks after the initial treatment. Subsequently, a relapse tendency to tumor proliferation was observed, despite a temporal suppression of tumor proliferation. In contrast, in the short-interval group (n=2), the tumor proliferation was sustainably suppressed and showed no relapse of tumor proliferation. It was confirmed that AMDC using Anti-HER2 VHH and Psyche-Duocarmycin suppressed tumor proliferation effectively and the intervals of additional treatment was essential to suppress the tumor proliferation sustainably.

<Pathological Analysis after In Vivo Experiment for Tumor Proliferation Suppressing Effect by the Interval Between Repeated Administrations in Xenograft Model Mouse>

Then, three mice in the long-interval group (n=3) and two mice in the short-interval group (n=2) were euthanized and pathologically examined 84 days after the initial treatment. The tumors were resected from the treated mice and fixed with 4% paraformaldehyde phosphate buffer solution (FUJIFILM Wako Pure Chemical Corporation) for 48 hours at 4° C., then embedded in paraffin following standard histopathological procedures. Histological specimens were deparaffinized and rehydrated by immersing them in xylene (FUJIFILM Wako Pure Chemical Corporation) for 10 min at room temperature, followed by successive immersions in 100, 90, 80, and 70% solutions of ethanol (FUJIFILM Wako Pure Chemical Corporation) for 3 min each at room temperature. Hematoxylin and eosin (Sakura Finetek Japan) solutions were used for H&E staining according to the manufacturer's protocols for dosage and administration. Finally, the stained slides were dehydrated via immersion in ethanol and xylene. The slides were covered with glass coverslips using Marinol (Muto Pure Chemicals) and histopathologically examined using the OLYMPUS cellSens Standard system (OLYMPUS). The results are shown in FIG. 14. In the short-interval group (n=2), infiltration of foamy macrophages, neutrophils, and other immune cells was observed with the formation of microscopic calcifications and cholesterol crystallization in these mice, as shown in #4 and #5 in FIG. 13. These characteristics are consistent with the post-necrotic pathological reactions observed upon the death of the tumor cells. No surviving cancer cells were identified. Both of two mice in the short-interval group had a complete tumor remission. In contrast, the tumor proliferation and recurrences were observed in the three mice in the long-interval group as shown in #1, #2, and #3 in FIG. 14, although in some tumor areas, innate immune cell infiltration, microscopic calcifications, and cholesterol crystallization were observed, suggesting the remnants of the cytotoxic activity as the treatment effect.

1. A fusion protein, wherein a VHH antibody is bound, via a linker sequence, to the N-terminal side and/or C-terminal side of the amino acid sequence as set forth in SEQ ID NO: 1, provided that the C-terminal amino acid sequence consisting of Pro-Ser-Ala-Ala-Ser-His-His-His-His-His-His may be partially or entirely deleted.

2. The fusion protein according to claim 1, which has the VHH antibody, the linker sequence, and the amino acid sequence as set forth in SEQ ID NO: 1, provided that the C-terminal amino acid sequence consisting of Pro-Ser-Ala-Ala-Ser-His-His-His-His-His-His may be partially or entirely deleted, in this order from the N-terminal side to the C-terminal side.

3. The fusion protein according to claim 1, wherein the VHH antibody is an antibody that binds to an antigen expressing in a cancer cell.

4. The fusion protein according to claim 1, wherein the VHH antibody is an antibody that binds to Her2.

5. The fusion protein according to claim 1, wherein the VHH antibody has the amino acid sequence as set forth in SEQ ID NO: 2.

6. The fusion protein according to claim 1, wherein the linker sequence consists of a glycine residue(s) and a serine residue(s) and the number of the amino acid residues is 5 to 25.

7. The fusion protein according to claim 1, wherein the linker sequence is an amino acid sequence represented by [(Gly)ₘ-Ser]ₙ wherein m represents an integer of 1 to 10, and n represents an integer of 1 to 5.

8. The fusion protein according to claim 1, which has the amino acid sequence as set forth in SEQ ID NO: 3, provided that the C-terminal amino acid sequence consisting of Pro-Ser-Ala-Ala-Ser-His-His-His-His-His-His may be partially or entirely deleted.

9. A nucleic acid encoding the fusion protein having the amino acid sequence as set forth in SEQ ID NO: 3, provided that the C-terminal amino acid sequence consisting of Pro-Ser-Ala-Ala-Ser-His-His-His-His-His-His may be partially or entirely deleted.

10. A cancer therapeutic agent or a cancer diagnostic agent, comprising the fusion protein according to claim 1.

11. A kit for treating or diagnosing cancer, comprising (1) the fusion protein according to claim 1, and (2) a conjugate of a compound represented by the following formula (1) or a salt thereof, and a diagnostic substance or a therapeutic substance: 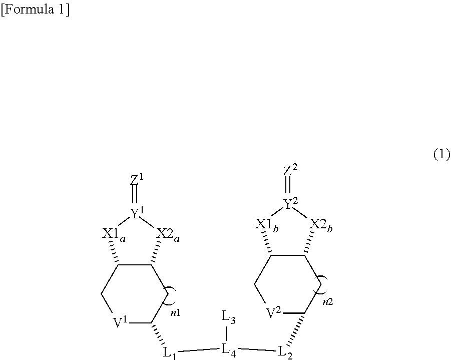
wherein
X1a, X1b, X2a and X2b each independently represent O or NH,
Y¹ and Y² each independently represent C or S,
Z¹ and Z² each independently represent O, S or NH,
V¹ and V² each independently represent S or S⁺—O⁻, n1 and n2 each independently represent an integer of 0 or 1,
L₁ and L₂ each independently represent a divalent linking group,
L₃ represents a group comprising a functional group capable of binding to the diagnostic substance or the therapeutic substance at the terminus thereof, and
L₄ represents a trivalent linking group.

12. The kit according to claim 11, wherein the diagnostic substance or the therapeutic substance is a phthalocyanine dye or an anti-cancer drug.

13. A method for producing the fusion protein according to claim 1, comprising
allowing a nucleic acid encoding the fusion protein to express in a host.

14. The method according to claim 13, wherein the fusion protein is allowed to express in a bacterial inclusion body and is then recovered.


SEQUENCE LISTING (8 sequences)

SEQ ID NO: 1 - protein, 133 aa, synthetic construct
  REGION 1..133: note = Description of Artificial Sequence: Recombinant protein
AEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 2 - protein, 116 aa, synthetic construct
  REGION 1..116: note = Description of Artificial Sequence: Recombinant protein
MQVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 3 - protein, 264 aa, synthetic construct
  REGION 1..264: note = Description of Artificial Sequence: Recombinant protein
MQVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSSGGGGSGGGGSGGGGSAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 4 - DNA, 789 bp, synthetic construct
  misc_feature 1..789: note = Description of Artificial Sequence: synthesized DNA
  source 1..789: mol_type = other DNA
caggttcagctgcaagaaagcggtggtggtagcgttcaggcaggcggtagcctgaaactgacctgtgcagcaagcggttatatctttaatagctgtggtatgggttggtatcgtcagagtccgggtcgtgaacgtgaactggttagccgtattagcggtgatggtgatacctggcataaagaaagcgttaaaggtcgttttaccatcagccaggataatgttaaaaagaccctgtacctgcagatgaatagtctgaaaccggaagataccgcagtgtatttttgtgccgtttgctataatctggaaacctattggggtcagggcacccaggttaccgttagcagcggaggcggagggtctggaggtggcggttcaggtggcggtggcagtgcggaagccggtattaccgggacctggtccgatcaactcggcgacacgtttatcgtgacagcaggcgcggatggcgcgctgacagggacgtacgagaatgcggtaggcggtgcggaaagccgctatgtgttgaccggtcgttacgattcggctccggctacggatggttccggtacagccttagggtggaccgttgcgtggaagaacaactcgaagaatgcccacagtgctaccacttggtcaggccagtatgttggcggggcggatgcgaaaatcaacactcagtggcttctgaccagcggaaccacgaatgccaatgcgtggaaatccacgctggtcggtcatgacacctttaccaaagtcaaaccgagtgcagcatcgcatcatcaccaccatcat

SEQ ID NO: 5 - DNA, 29 bp, synthetic construct
  misc_feature 1..29: note = Description of Artificial Sequence: Synthesized DNA
  source 1..29: mol_type = other DNA
catggtatatctccttcttaaagttaaac

SEQ ID NO: 6 - DNA, 26 bp, synthetic construct
  misc_feature 1..26: note = Description of Artificial Sequence: Synthesized DNA
  source 1..26: mol_type = other DNA
taacgcagcttaattaacctaggctg

SEQ ID NO: 7 - DNA, 40 bp, synthetic construct
  misc_feature 1..40: note = Description of Artificial Sequence: Synthesized DNA
  source 1..40: mol_type = other DNA
aggagatataccatgcaggttcagctgcaagaaagcggtg

SEQ ID NO: 8 - DNA, 41 bp, synthetic construct
  misc_feature 1..41: note = Description of Artificial Sequence: Synthesized DNA
  source 1..41: mol_type = other DNA
aattaagctgcgttaatgatggtggtgatgatgcgatgctg